(19) United States
(12) Patent Application Publication
(10) Pub. No.: US 2025/0258545 A1
(43) Pub. Date: Aug. 14, 2025

(54) 3D Digital Immortality And Uses Thereof

(71) Applicant: West Nanorobotics LLC, Pendleton, IN (US)

(72) Inventor: Tory Cortland Weston, Pendleton, IN (US)

(73) Assignee: West Nanorobotics LLC, Pendleton, IN (US)

(21) Appl. No.: 18/438,472

(22) Filed: Feb. 11, 2024

(51) Int. Cl. G06F 3/01

(52) U.S. Cl. CPC G06F 3/015

(57) ABSTRACT

This is a method for making digital emulations of human brains by mind uploading the consciousness of the person into a computer by usage of magnetic resonance imaging (M.R.I.) scans and electroencephalogram (E.E.G.) Scans of the human brain. The process makes a exact digital copy of the brain in the computer which acts just as the organic human brain using arrays and tensors being a digital form of the human brain and the exact electrical processes that happen in the organic human brain happen in the digital emulation allowing the exact same mental processes to happen within the digital environment of the computer. The digital emulation is then processed inside a robotic body of the host allowing the host to achieve control over the robotic body and a form of digital immortality as a infomorph.

### DESCRIPTION

### Brief Summary

The process begins as the structural data and electrical data are obtained by Magnetic Resonance Imaging and Electroencephalogram scans of the human brain. This allows the computer to obtain critical data about the human brain used in the emulation of the human brain inside the computer. The information is then stored inside a 3-D array called a Tensor with a equal number of points in the tensor to the number of neurons in the human brain with a number within each point that measures the electrical charge of that neuron at the exact moment of the Electroencephalogram scan of the human brain. The Tensor is then given structure based on the Magnetic Resonance Imaging scan of the human brain being that the Tensor is formed in the exact same pattern as the scan being in the same shape as the organic human brain counterpart. The saved firing pattern being the electrical activity is then processed by the emulation to continue at the same period of time when the scans were taken allowing for the exact thought processes and stored information being memories to continue from that point in time within the emulation. Lastly, the neurotransmitters of the brain continue to be released within the emulation as emulated neurotransmitters being just numbers which alter the electrical activity from the moment the scans were taken providing the same functionality in the emulation as the organic human brain counterpart making a exact copy of the human consciousness if he process was followed perfectly. The emulation is then connected to a robotic spinal column and the motor functions happen just as the organic human body in the robotic body of the host.

### BRIEF DESCRIPTION OF THE FIGURES

FIG. 1, The Diagram of a Emulation Tensor overlapped with the M.R.I. Scan.

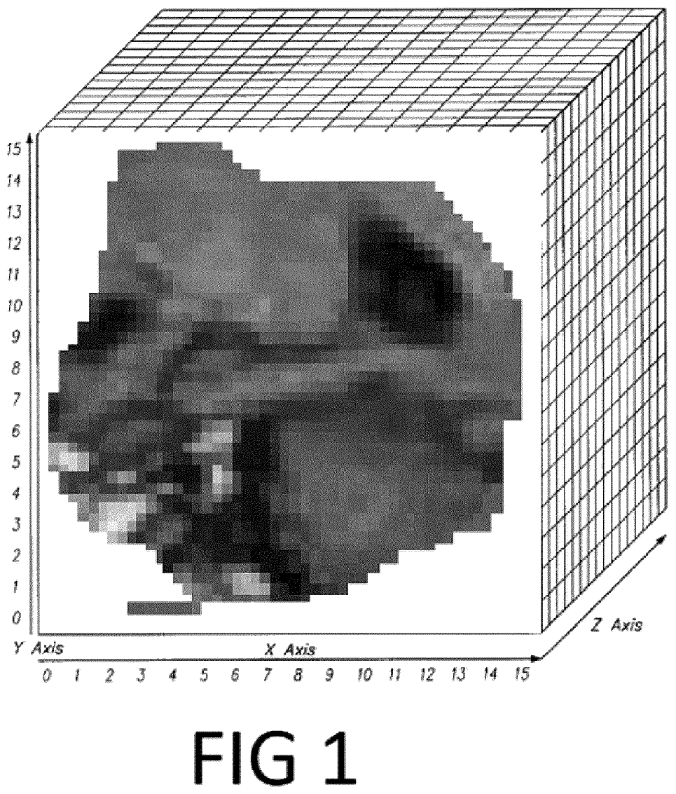

FIG. 2, The Diagram of the Emulation Tensor in a Digital Environment.

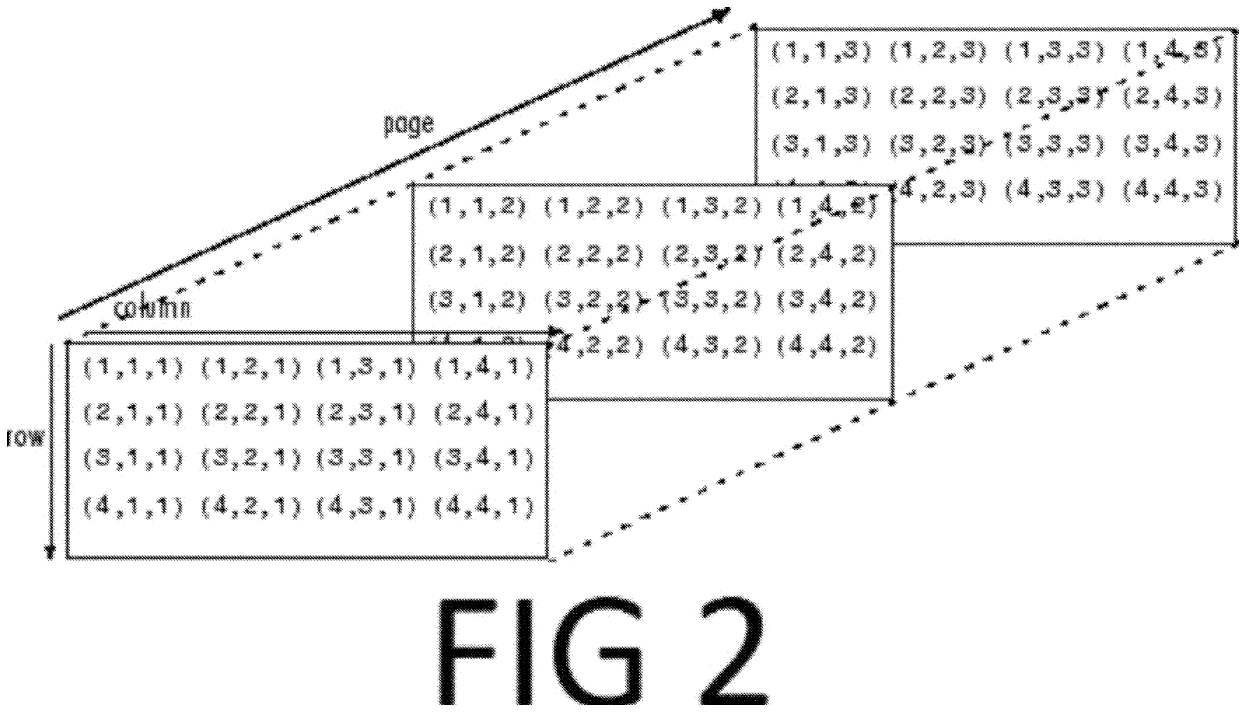

### DETAILED DESCRIPTION

This process happens in several key steps which will be outlined in the following order, Electroencephalogram Scan, Magnetic Resonance Imaging Scan, Emulation Tensor, Emulation Tensor Shaping, Electrical Emulation, and Neurotransmitter Emulation.

First, the Electroencephalogram must be taken while the host is still alive reading the electrical activity of each individual neuron taking the “Mind Data” of the organic human brain. The resolution of this scan must be smaller than 100 microns to read the information in the form of electrical impulses from the human brain which means attaching electrical monitoring equipment inside the human brain in the form of microchips inside the brain which would probably be a form of nanorobotics such as medical nanorobots using these medical nanorobots the Electroencephalogram is taken of each neuron much like a normal Electroencephalogram scan but with higher resolution and three dimensionally. The information from the scan is stored in a excel file as readings of the charge of the neurons of the human brain to later be used in the Emulation Tensor. These numbers will be a charge value in Coulombs and there should be a single reading at the same exact time for each neuron in the human brain otherwise if all neurons not taken within five milliseconds of each other than “Mind Data” will not reflect the actual “Mind Data” of the organic human brain and will need to be retaken until the neurons are all taken within five milliseconds of each other.

Second, the Magnetic Resonance Imaging Scan must be detailed to the point that each individual synapse can be registered in the scan to a scale of 2 microns in resolution. The entire structure of the human brain must be scanned three dimensionally which means cutting the brain into slices being a single neuron in thickness or 100 microns. This magnetic resonance imaging scan will need to also read the connections of the neurons to each other measuring the structure of each synapse showing the connections of the neurons to each other. This process should happen after the host has died by natural means but before it has decayed too much as soon as possible after death within at least three hours. This should also happen after the Electroencephalogram Scan which requires the host body to be alive to register the electrical activity to gather the “Mind Data” of the human brain. The Magnetic Resonance Imaging scan should be taken of each slice and stored in the form of a three dimensional image which later will be used in the Emulation Tensor Shaping part of the process to shape the Emulation Tensor in the exact shape of the structural Magnetic Resonance Imaging Scan making a duplicate of the organic human brain's structure down to each individual synapse.

Third, the Emulation Tensor which is a type of 3 dimensional array that stores the data of the electrical activity to be processed which is taken from the excel document of electrical activity being the “Mind Data” of the organic human brain. The “Mind Data” is measured in Coulombs which these numbers need to be input into the Emulation Tensor which each point within the Emulation Tensor corresponds to a single neuron that the charge measurement was taken from. There are a set of around 86 billion array points in the Emulation Tensor each with a number of charge in Coulombs. This is the essence of the organic brain's “Mind Data” which the Emulation Tensor will continue to process the information at the exact moment the Electroencephalogram was taken in the Emulation Tensor which the Emulation Tensor Shaping process will establish connections between the points in the Emulation Tensor allowing for the Emulation Tensor to process this “Mind Data” which the Emulation Tensor will change in the number values of the charge as the “Mind Data” continues to process within the Emulation Tensor as it runs within the robotic body of the host or a computer allowing for consciousness and thought to happen within the Emulation Tensor just as a organic human brain.

Fourth, the Emulation Tensor Shaping which will form the Emulation Tensor to the shape of the magnetic resonance imaging scan previously taken establishing the exact same connections between points in the Emulation Tensor as the synapses did in the organic human brain. The shape of the Emulation Tensor will be that of a human brain after a computer program sets the Emulation Tensor position numbers in (X, Y, Z) coordinates which will be (X, Y, Z) coordinates taken from the magnetic resonance imaging scan which was previously mentioned. This magnetic resonance imaging scan measured the synaptic connections of the organic human brain and the exact same connections will be made that were in the organic human brain in the emulation tensor to allow the Emulation Tensor to act as the organic human brain did with the same functionality.

Fifth the Electrical Emulation, As a point in the Emulation Tensor discharges in the virtual environment starting with the original “Mind Data” thus will the points connected to that point receive that discharge and begin to make alterations in numerical form to themselves because of the discharge which is all carried out as the charge numbers stored within the point which act as artificial neurons using a computer algorithm before transmitting it to the connected points. This will allow for processing of the “Mind Data” and it to act as the organic human brain did just in a virtual environment of numbers making consciousness and thought occur within the Emulation Tensor of the computer or robotic body of the host. This is this information being taken from the original organic human brain and processed just as the organic human brain being also shaped in the structure along with connected in the same form as the organic human brain being a exact duplicate of the organic human brain.

Sixth, Neurotransmitter Emulation, the neurotransmitters will activate or deactivate a response in the points just as they would neurons it is known from neuroscience which parts of the brain have receptors for which types of neurotransmitters and which cells release said neurotransmitters this will be added the the emulation as a set of numbers which will take the values of zero or one depending on if each type of receptor is present. These numbers will be each another variable within the Emulation Tensor for each point each variable corresponding to a certain neurotransmitter receptor and a algorithm will calculate the laws of physics to make a accurate model of the movement of the neurotransmitters through the Emulation Tensor as if it were a organic human brain calculating such things as density of the neurotransmitter and which points receive them within the Emulation Tensor in such a way were it does them for all neurotransmitters present in a organic human brain.

This makes the digital immortality process possible these steps; the emulation of all the functionality of the organic human brain within a virtual environment as purely data in the form of complex algorithms to manipulate it which a accurate to reality which is taken from a real living human brain.

### How to Use

The emulation tensor will need to be hosted inside a computer or robotic body with enough processing power to handle the calculations required and a virtual spinal column will need to be attached to the emulation tensor with moves the actual robotic body of the host. The person will think and have consciousness within the computer or robotic body allowing for digital immortality as a infomorph being a construct at this point of pure “Mind Data”. The Emulation Tensor will automatically run in a loop processing the “Mind Data” based on the template gathered from the magnetic resonance imaging scan and electroencephalogram scan hosting the living mind of a human being.

1. Any Digital Emulation of the human brain especially those using Tensors or Arrays to store the information of the human brain.

2. Any Mind Uploading Process using Magnetic Resonance Imaging (M.R.I.) Scans and Electroencephalogram (E.E.G.) Scans to bring the information of the human brain into a digital environment especially those using a combination of both methods to obtain the structure of the human brain and electrical activity of the human brain.

3. Any usage of nanorobotics to collect the electrical activity of the human brain especially self replicating nanofactories.
